(19) United States
(12) Patent Application Publication
(10) Pub. No.: US 2019/0105215 A1
(43) Pub. Date: Apr. 11, 2019

(54) CONTROL SYSTEM UTILIZING A MOBILE APPLICATION FOR A LEGGED MOBILITY EXOSKELETON DEVICE

(71) Applicant: Parker-Hannifin Corporation, Cleveland, OH (US)

(72) Inventors: Skyler Ashton Dalley, Shaker Heights, OH (US); Ryan Farris, Hartville, OH (US); Scott Morrison, Mount Pleasant, MI (US)

(21) Appl. No.: 15/743,136

(22) PCT Filed: Jun. 30, 2016
(86) PCT No.: PCT/US2016/040304

§ 371 Date: Jan. 9, 2018

Related U.S. Application Data
(60) Provisional application No. 62/266,787, filed on Dec. 14, 2015.

(51) Int. Cl. A61H 1/02; A61N 1/36; B25J 9/00; A61H 3/00; G16H 20/30

(52) U.S. Cl. CPC A61H 1/024; A61H 2201/165; A61H 1/0244; B25J 9/0006; A61H 3/00; G16H 20/30; A61H 2230/305; A61H 2230/203; A61H 2230/065; A61H 2201/5097; A61H 2201/5084; A61H 2201/5079; A61H 2201/0188; A61H 2201/5048; A61H 2201/5069; A61H 2201/501; A61H 2201/5043; A61H 2201/0192; A61H 2201/1207; A61H 2201/1463; A61H 2201/1628; A61H 2201/164; A61N 1/36003

(57) ABSTRACT

A method of controlling a mobility device including at least one drive component that drives at least one joint component is described. The control may include providing said mobility device, providing an electronic communication device having a control application to be executed by the electronic control device, receiving an input of settings information to the electronic communication device, the settings information being stored by the control application, electronically connecting the electronic communication device to the mobility device, and executing the control application with the electronic communication device to perform a session of using the mobility device. The electronic communication device executes the control application to control the at least one drive component of the mobility device to selectively configure and modulate the at least one joint component in accordance with the settings information. The control application may be based on multiple device and/or user profiles with the settings being set based on the profiles. Session information may be displayed in real time as a displayed session dashboard, and stored in session logs for future review and analysis.

### RELATED APPLICATIONS

This application claims the benefit of U.S. Provisional Application No. 62/226,787 filed Dec. 15, 2015, which is incorporated herein by reference.

### FIELD OF INVENTION

The present invention relates to electronic control systems for a legged mobility device or “exoskeleton” device, including control systems for unlocking or otherwise enabling, configuring, and observing the internal state of or other events for the legged mobility device or exoskeleton device.

### BACKGROUND OF THE INVENTION

There are currently on the order of several hundred thousand spinal cord injured (SCI) individuals in the United States, with roughly 12,000 new injuries sustained each year at an average age of injury of 40.2 years. Of these, approximately 44% (approximately 5300 cases per year) result in paraplegia. One of the most significant impairments resulting from paraplegia is the loss of mobility, particularly given the relatively young age at which such injuries occur. Surveys of users with paraplegia indicate that mobility concerns are among the most prevalent, and that chief among mobility desires is the ability to walk and stand. In addition to impaired mobility, the inability to stand and walk entails severe physiological effects, including muscular atrophy, loss of bone mineral content, frequent skin breakdown problems, increased incidence of urinary tract infection, muscle spasticity, impaired lymphatic and vascular circulation, impaired digestive operation, and reduced respiratory and cardiovascular capacities.

In an effort to restore some degree of legged mobility to individuals with paraplegia, several lower limb orthoses have been developed. The simplest form of such devices is passive orthotics with long-leg braces that incorporate a pair of ankle-foot orthoses (AFOs) to provide support at the ankles, which are coupled with leg braces that lock the knee joints in full extension. The hips are typically stabilized by the tension in the ligaments and musculature on the anterior aspect of the pelvis. Since almost all energy for movement is provided by the upper body, these passive orthoses require considerable upper body strength and a high level of physical exertion, and provide very slow walking speeds.

The hip guidance orthosis (HGO), which is a variation on long-leg braces, incorporates hip joints that rigidly resist hip adduction and abduction, and rigid shoe plates that provide increased center of gravity elevation at toe-off, thus enabling a greater degree of forward progression per stride. Another variation on the long-leg orthosis, the reciprocating gait orthosis (RGO), incorporates a kinematic constraint that links hip flexion of one leg with hip extension of the other, typically by means of a push-pull cable assembly. As with other passive orthoses, the user leans forward against a stability aid (e.g., bracing crutches or a walker) while un-weighting the swing leg and utilizing gravity to provide hip extension of the stance leg. Since motion of the hip joints is reciprocally coupled through the reciprocating mechanism, the gravity-induced hip extension also provides contralateral hip flexion (of the swing leg), such that the stride length of gait is increased. One variation on the RGO incorporates a hydraulic-circuit-based variable coupling between the left and right hip joints. Experiments with this variation indicate improved hip kinematics with the modulated hydraulic coupling.

To decrease the high level of exertion associated with passive orthoses, the use of powered orthoses has been under development, which incorporate actuators and drive motors associated with a power supply to assist with locomotion. These powered orthoses have been shown to increase gait speed and decrease compensatory motions, relative to walking without powered assistance.

The use of powered orthoses presents an opportunity for electronic control of the orthoses. Exoskeleton devices to date, however, have lacked comprehensive control systems that also are user-friendly to maximize the effectiveness and comfort for a legged exoskeleton device.

Examples of powered orthoses are known. WO/2010/044087, US 2010/0094188, and U.S. Pat. No. 8,096,965 disclose a powered exoskeleton bracing system/exoskeleton bracing system. These prior art devices, however, have been insufficient for comprehensive and user-friendly control of the exoskeleton device. The conventional methods of user interfacing with control of the device tended to focus on safety features to generate alerts that tend to be in response to a defective nature or state of the exoskeleton device or its components. Alerts, for example, may be provided as to such conditions as sensor fault(s), detection of “high” temperature(s), detection of battery charger or battery malfunction (High Severity Alerts accompanied by Solid Red LEDs), detection of “medium” temperature(s), detection of “critical” battery levels (Medium Severity Alerts accompanied by Flashing Red LEDs), detection of low temperature(s), and detection of “low” battery levels (Low Severity Alerts accompanied by Flashing Yellow LEDs).

There have been attempts to provide at least generalized control of an exoskeleton device. For example, U.S. Pat. No. 8,348,875 discloses a method of controlling an exoskeleton bracing system to walk forward comprising operating an alerting device to generate an alert in response to a sensed condition, wherein the sensed condition comprises falling. U.S. Pat. No. 8,905,955 B2 discloses a method of controlling an exoskeleton bracing system comprising halting actuation of the motorized joints when a signal that is received from a tilt sensor indicates falling. These methods are described entirely within the context of standing and sitting transitions.

WO/2010/044087, US 2010/0094188, and U.S. Pat. No. 8,096,965 disclose an exoskeleton bracing system/exoskeleton bracing system control method mode selector for selecting the mode of locomotion from a predefined set of options. The mode selector can connect wirelessly to the device and straps to the wrist of the user. The predefined set of operation modes includes walking, standing, sitting, and stair climbing.

WO/2012/052988 and US 2012/0101415 disclose a locomotion assisting exoskeleton device include a remote control device. WO/2013/142777 discloses a method of controlling a powered lower extremity orthotic, wherein the leg support includes a thigh segment and a shank segment. The control method includes estimating an angle of the shank segment with respect to vertical, and taking a step when the shank angle exceeds a threshold with respect to gravity. The control operations further may include signaling the user when placing the orthotic into a state corresponding to taking a step, as accomplished by an auditory tone, haptic vibration, or visual cue.

WO/2014/159577 discloses a lower extremity orthosis configured to be coupled to a person, and a controller that receives signals from a plurality of sensors. The controller estimates at least one feedback ready value based on the sensor output, and at least one feedback system operated by the controller is configured to communicate the feedback ready value to the user. The orthosis provides the person with orthosis operational information not otherwise available to the user, wherein the feedback systems includes at least one light indicating actuator effort, a plurality of lights proportionally indicating actuator torque, at least one light indicating force at an interface point, a plurality of lights proportionally indicating force at an interface point. The feedback ready value is selected from: forced between person and orthosis, effort applied by orthosis, torque applied by orthosis, maximum effort applied over gait cycle, average effort applied over gait cycle, center of pressure, limb position, center of mass position, foot clearance, orthosis state, next orthosis action, optimal gain aid orientation, and movement of the person.

FIGS. 2 and 3 of WO/2014/159577 show a therapist holding a “control input means” (in this case what appears to be a tablet computer). However, as stated in paragraph [0057] of the description, “input devices are not a particular object of this invention.” Furthermore, it can be seen from FIGS. 2 and 3 and their accompanying descriptions that the indicators are to be located on the exoskeleton itself, and not on the input device. In paragraph [0093], alternative locations for the indicators are mentioned in reference to FIG. 11, but none of the proposed locations, nor the figure itself, indicate the input device as being a location of the indicators. That is, the feedback system described in this application primarily consists of discrete hardware (lights or other transducers) which are located on the exoskeleton, and are considered separately from the control input device throughout the description.

The conventional control systems described above have limited scope, and they tend to be complex and not user friendly. Typically, such interfaces require dedicated hardware and are used primarily for mode or state transitions, and have relatively limited capability. Convention communicator systems representative of the state of the art may employ a wireless interface worn on the wrist of the user that is primarily used to implement mobility mode transitions (e.g., Sit to Stand, Stand to Walk, Walk to Stand, Stand to Sit, etc.). Such communicator will also output warning of a wireless communication failure or provides battery indications for both the exoskeleton device and the communicator interface device. Another conventional system may be implemented as a controller with a wired interface held in the hand that similarly is primarily used to implement mobility mode transitions (e.g., Sit to Stand, Stand to Walk, Walk to Stand, Stand to Sit, etc.) and step transition (e.g., Right Step, Left Step). The wired controller also may warn of battery, sensor, actuator, software, hardware, and transition errors.

Although conventional control systems exist that implement mode transitions and output warnings, the scope of such control encompasses only a small fraction of potential operational modes and use cases for an exoskeleton device. Control systems for exoskeleton devices to date, therefore, have lacked comprehensive control systems that also are user-friendly to maximize the effectiveness and comfort for a legged mobility exoskeleton device.

### SUMMARY OF THE INVENTION

The present invention is directed to control methods and a related control system that is configured to enable and configure a mobility device, such as for example a legged mobility exoskeleton device, through a mobile application in which multiple profiles representing users or use cases may be created. Each profile may store any of the following categories of information, in any combination: user information (e.g., height/weight), device information (e.g. sizing/configuration), device settings (e.g. step length/step speed), device data (e.g. steps taken), or session logs, a “session” being defined as any combination of the above categories of information grouped according to a specific time period of device use. A session may be an active or passive session pertaining to use of the exoskeleton device. For example, a passive session may be merely a time period of gathering information about a device state, configuring settings, and otherwise monitoring or effecting a status or condition of an exoskeleton device. An active session may employ a user operating the exoskeleton device for mobility (e.g., sitting, walking, standing). A session further may include both active and passive aspects in any combination.

Within each profile, device settings may be grouped according to various mobility modes, such as for example sitting, standing, walking, or others, which are implemented in accordance with the device settings. The device settings may be transmitted to the exoskeleton device when a wireless connection is made, such as at the start of a session, which both may enable the device and further configures the exoskeleton device for a given profile. Device settings may also be changed while the device is in session, with some settings being queued for later implementation so as to not disrupt motion or present hazards (for example, step speed cannot be changed in the middle of a step).

The control system and methods of the present invention also allow a legged mobility device internal data to be observed. During a session, a mobile application that may be installed on any suitable portable electronic device may display information relating to the device both as a virtual instrument panel, or “dashboard”, (e.g. battery level, session duration), and/or as a series of discrete session events (e.g. Session Started, Device Standby). The Profiles and Session Logs may be exported for record storage and future use.

In contrast to the conventional control systems described above, the control system and methods of the present invention provide an interface apparatus that uses a mobile application that goes beyond mode selection for stand, walk, and sit modes, or to provide alerts. Rather, in the exoskeleton device designed by the current inventors and their colleagues, mode selection is performed automatically by the user via postural cues that are sensed by the exoskeleton device itself. Accordingly, the control system of the present invention is capable of modifying the behavior or settings of the exoskeleton at a more detailed level within these modes that are entered by the sensed postural cues. For example, the control system described in this application may indicate when a step has occurred, but does so after the fact insofar as stepping is performed automatically by the sensed postural cues by the exoskeleton device. Additional control of stepping may be provided by the user input for enhanced performance, such as by setting stepping speed, stride length, or the like. Such more precise control within any given mode is beyond the scope of conventional systems. The control system thus includes an electronic application that can connect wirelessly through a mobile electronic communication device, but need not be worn by the user (such as on the wrist) as required in conventional systems.

A mobile electronic control application executed in accordance with present invention does not need to run on any dedicated or specialized hardware, but may run on any suitable electronic communication device, with mobile or portable electronic devices being most convenient for a user. Because of such versatility, the control application may be password protected, and used to enable the legged mobility or exoskeleton device, thus restricting access to application information and device function. Profiles may be created for each device User or Use Case, such that a given device may be rapidly configured for a given user or situation. These user configurations may be implemented by executing the control application to establish device settings that are deliberately grouped into mobility modes, so that the application operator can find them quickly and intuitively. Device settings may be changed prior to exoskeleton device use or while the exoskeleton device is in operation. During an operational session, information may be presented to the user as both continuous values within a graphical dashboard, and/or as a series of discrete events within an ordered list. Information stored in or collected by the application can then be transmitted or collected for past record or future use. In this context, a user may be the wearer of the device, or may be another party such as a caregiver who may be monitoring the wearer and device performance.

These and further features of the present invention will be apparent with reference to the following description and attached drawings. In the description and drawings, particular embodiments of the invention have been disclosed in detail as being indicative of some of the ways in which the principles of the invention may be employed, but it is understood that the invention is not limited correspondingly in scope. Rather, the invention includes all changes, modifications and equivalents coming within the spirit and terms of the claims appended hereto. Features that are described and/or illustrated with respect to one embodiment may be used in the same way or in a similar way in one or more other embodiments and/or in combination with or instead of the features of the other embodiments.

### BRIEF DESCRIPTION OF THE DRAWINGS

FIG. 1 is a drawing depicting an exemplary exoskeleton device as being worn by a user.

FIG. 2 is a drawing depicting a perspective view of an exemplary exoskeleton device in a standing position.

FIG. 3 is a drawing depicting a perspective view of the exemplary exoskeleton device in a seated position.

FIG. 4 is a drawing depicting a front view of the exemplary exoskeleton device in a standing position.

FIG. 5 is a drawing depicting a side view of the exemplary exoskeleton device in a standing position.

FIG. 6 is a drawing depicting a back view of the exemplary exoskeleton device in a standing position.

FIG. 7 is a drawing depicting a perspective view of an exemplary thigh assembly having two exemplary actuator cassettes installed therein.

FIG. 8 is a drawing depicting a front exploded view of the exemplary thigh assembly having two exemplary actuator cassettes installed therein.

FIG. 9 is a drawing depicting a perspective exploded view of the exemplary thigh assembly having two exemplary actuator cassettes installed therein.

FIG. 10 is a drawing depicting a top view of an exemplary actuator cassette.

FIG. 11 is a drawing depicting a bottom view of an exemplary actuator cassette.

FIG. 12 is a drawing depicting a perspective view of an exemplary actuator cassette.

FIG. 13 is a drawing depicting a cross-sectional view of an exemplary actuator cassette taken along the longitudinal direction.

FIG. 14 is a drawing of a generalized depiction of an exemplary mobile communication device interacting with the exoskeleton device of FIG. 1.

FIG. 15 is a schematic block diagram depicting operative portions of an exemplary mobile communication device for use in accordance with embodiments of the present invention.

FIG. 16 is a drawing depicting an exemplary screenshot for a user login screen in accordance with embodiments of the present invention.

FIG. 17 is a drawing depicting an exemplary screenshot for a user accounts screen in accordance with embodiments of the present invention.

FIG. 18 is a drawing depicting an exemplary screenshot for a user profile screen in accordance with embodiments of the present invention.

FIG. 19 is a drawing depicting an exemplary screenshot for a profile detail screen in accordance with embodiments of the present invention.

FIG. 20 is a drawing depicting an exemplary screenshot for a device settings screen in accordance with embodiments of the present invention.

FIG. 21 is a drawing depicting an exemplary screenshot for a session dashboard screen in accordance with embodiments of the present invention.

FIG. 22 is a drawing depicting an exemplary screenshot for a session log screen for an individual session in accordance with embodiments of the present invention.

FIG. 23 is a drawing depicting an exemplary screenshot for a session details screen for an individual session in accordance with embodiments of the present invention.

FIG. 24 is a drawing depicting an exemplary screenshot for a session log export screen for an individual session in accordance with embodiments of the present invention.

### DETAILED DESCRIPTION

Embodiments of the present invention will now be described with reference to the drawings, wherein like reference numerals are used to refer to like elements throughout. It will be understood that the figures are not necessarily to scale.

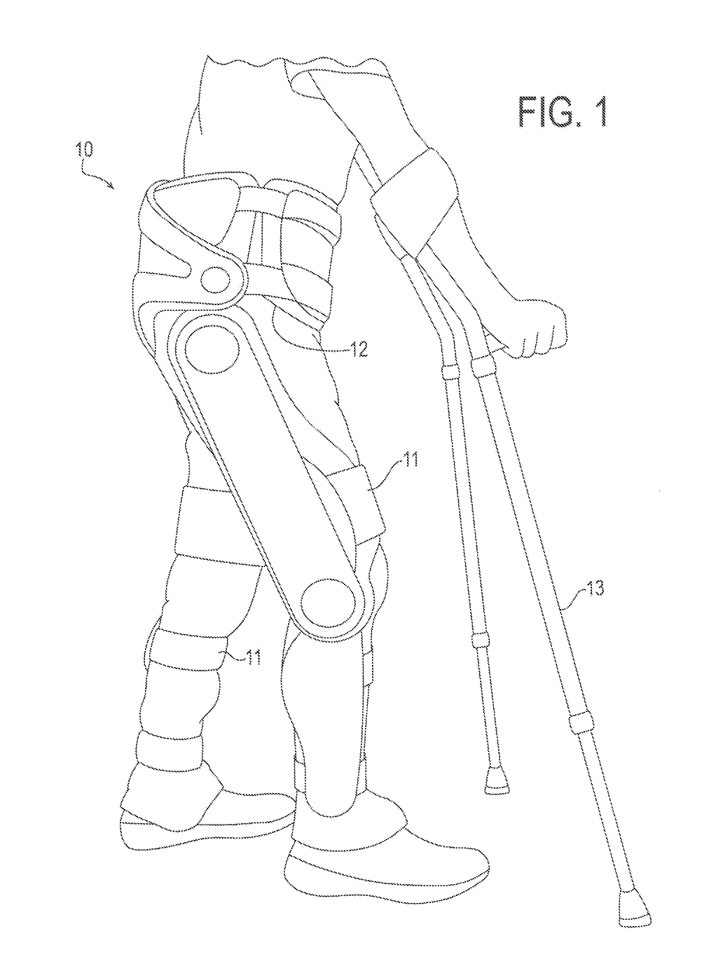

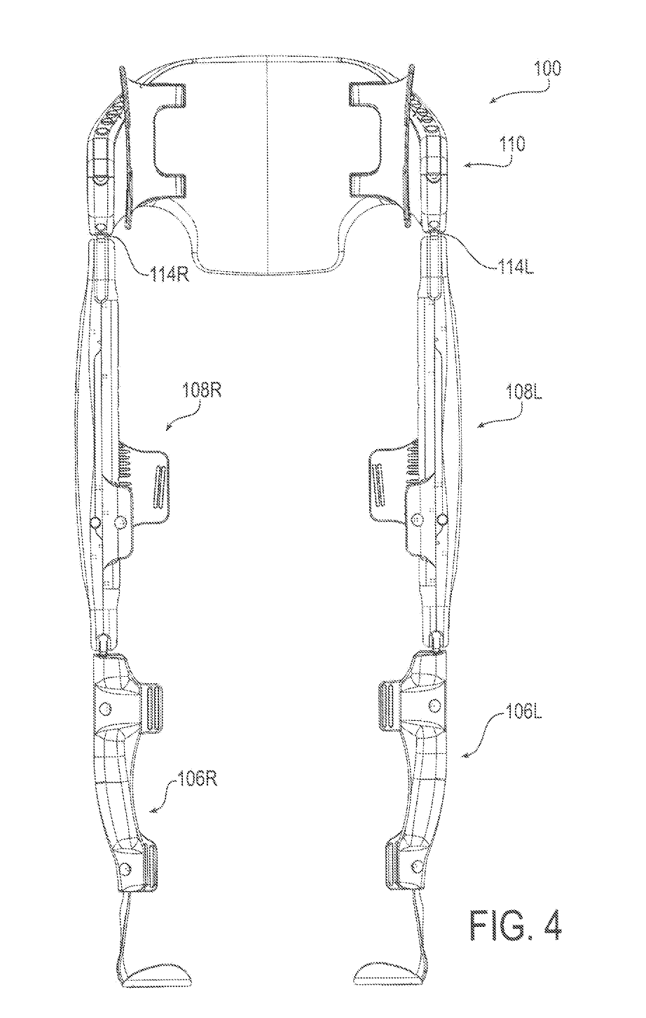

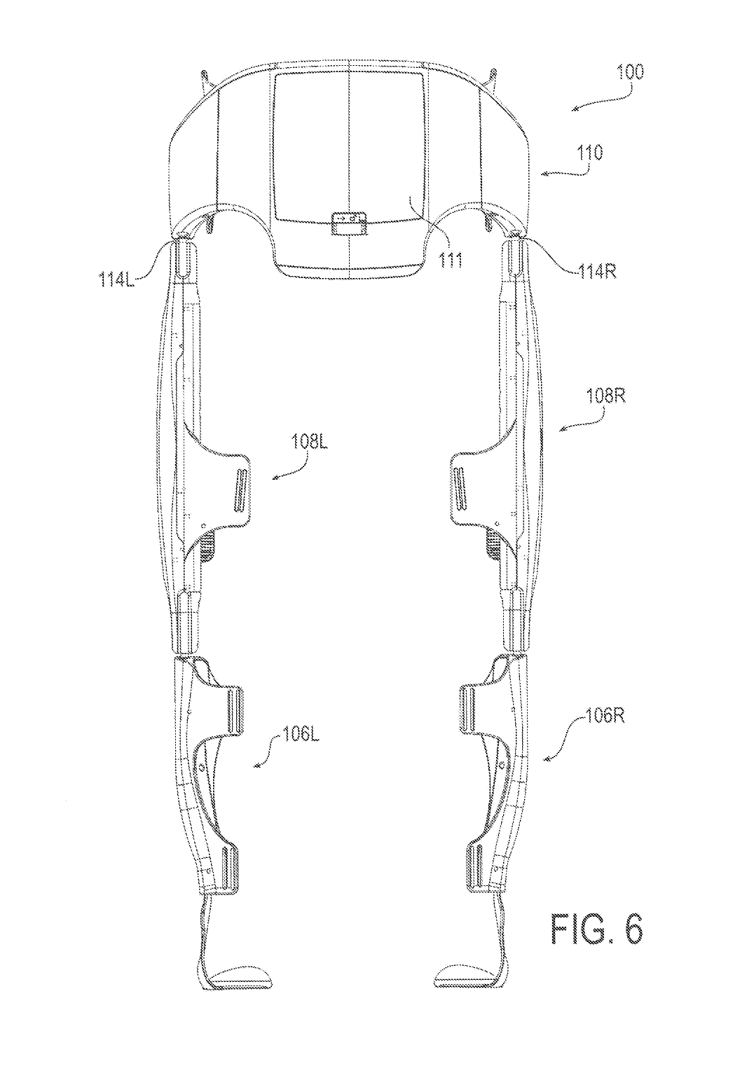

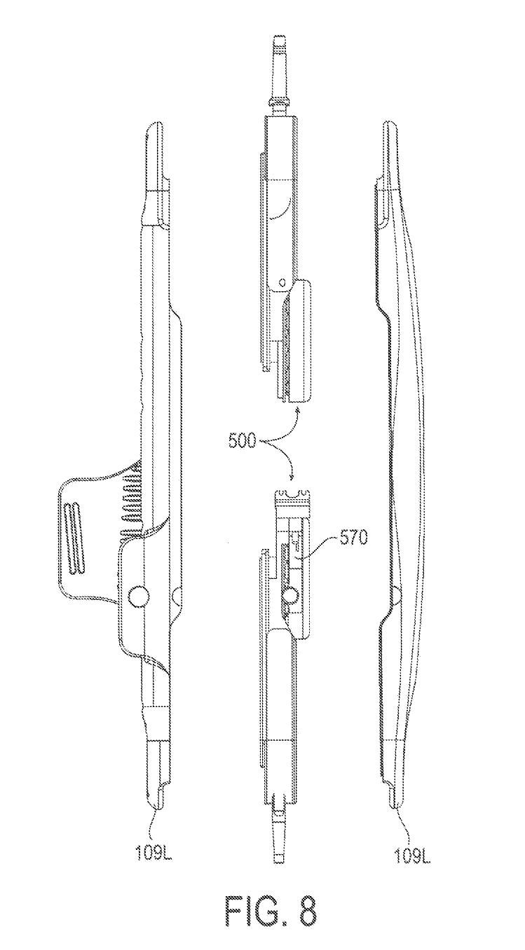

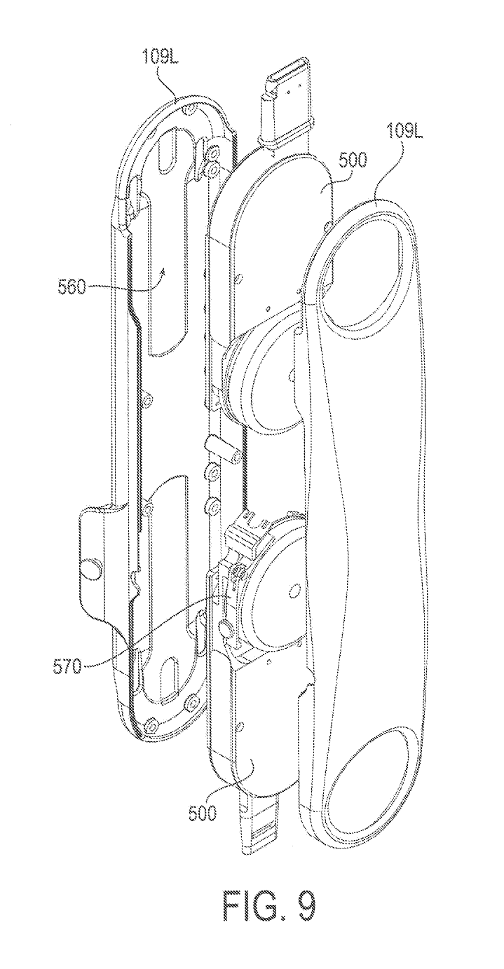

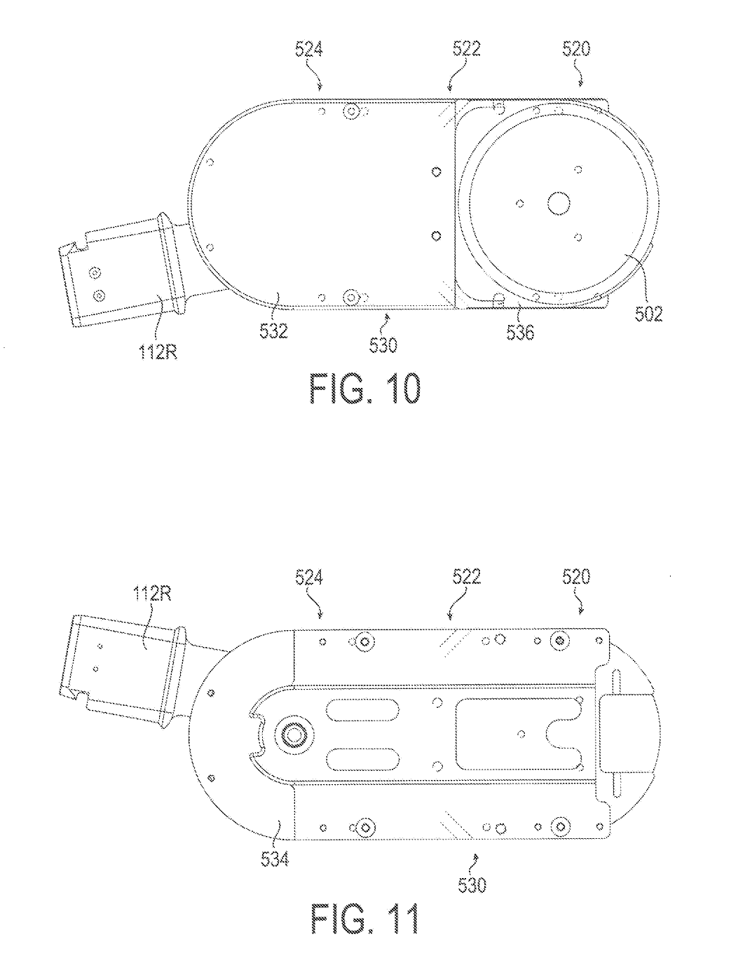

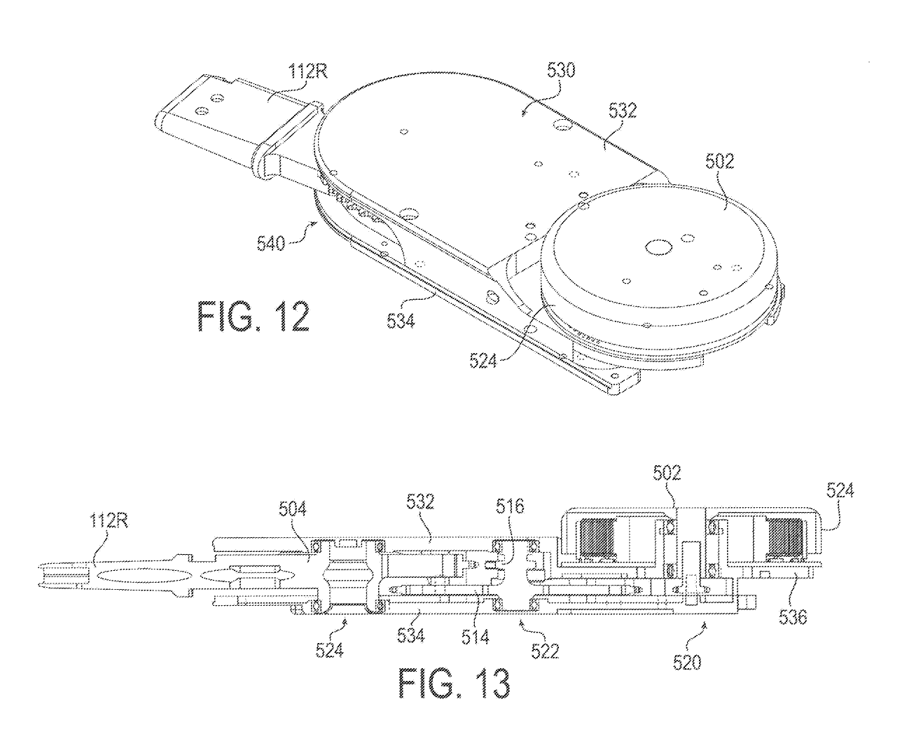

For context, FIGS. 1-13 depict various views of an exemplary exoskeleton device that may be used in connection with the control system and methods of the present invention. A somewhat generalized description of such exoskeleton device is provided here for illustration purposes. A more detailed description of such device may be found in Applicant's International Patent Appl. No. PCT/US2015/023624 filed on Mar. 3, 2015, which is incorporated here in its entirety by reference. It will be appreciated, however, that the described exoskeleton device presents an example usage, and that the control system and methods of the present invention are not limited to any particular configuration of an exoskeleton device. Variations may be made to the exoskeleton device, while the features of the present invention remain applicable. In addition, the principles of this invention may be applied generally to any suitable mobility device. Such mobility devices include, for example, orthotic devices which aid in mobility for persons without use or limited use of a certain body portion, and prosthetic devices, which essentially provide an electro-mechanical replacement of a body part that is not present such as may be used by an amputee or a person congenitally missing a body portion.

As show in FIG. 1, an exoskeleton device 10, which also may be referred to in the art as a “wearable robotic device”, can be worn by a user. To attach the device to the user, the device 10 can include attachment devices 11 for attachment of the device to the user via belts, loops, straps, or the like. Furthermore, for comfort of the user, the device 10 can include padding 12 disposed along any surface likely to come into contact with the user. The device 10 can be used with a stability aid 13, such as crutches, a walker, or the like.

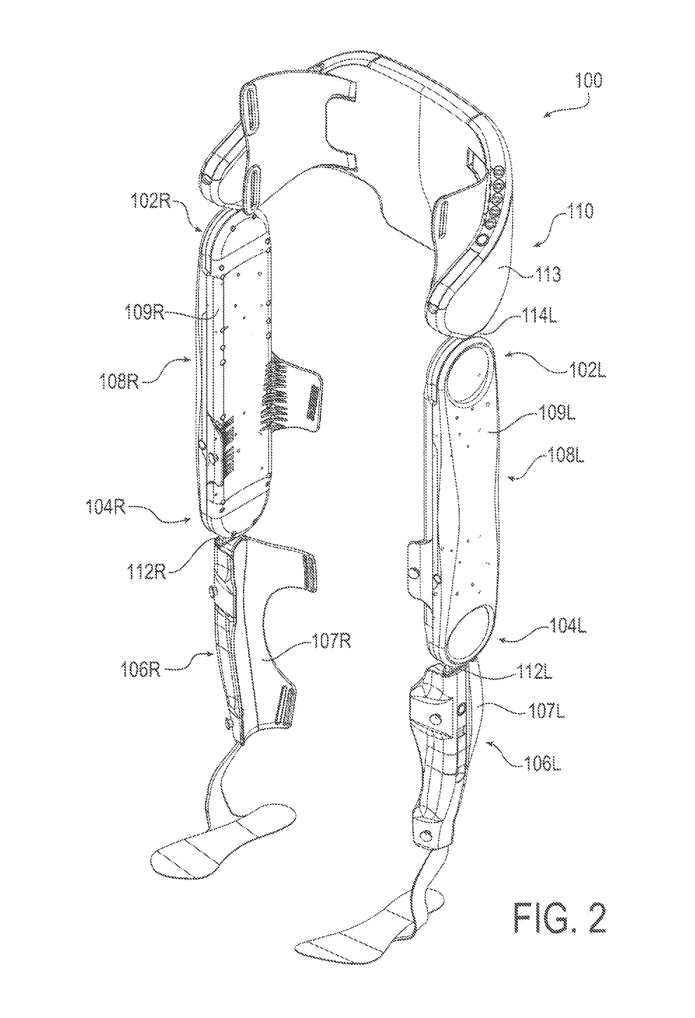

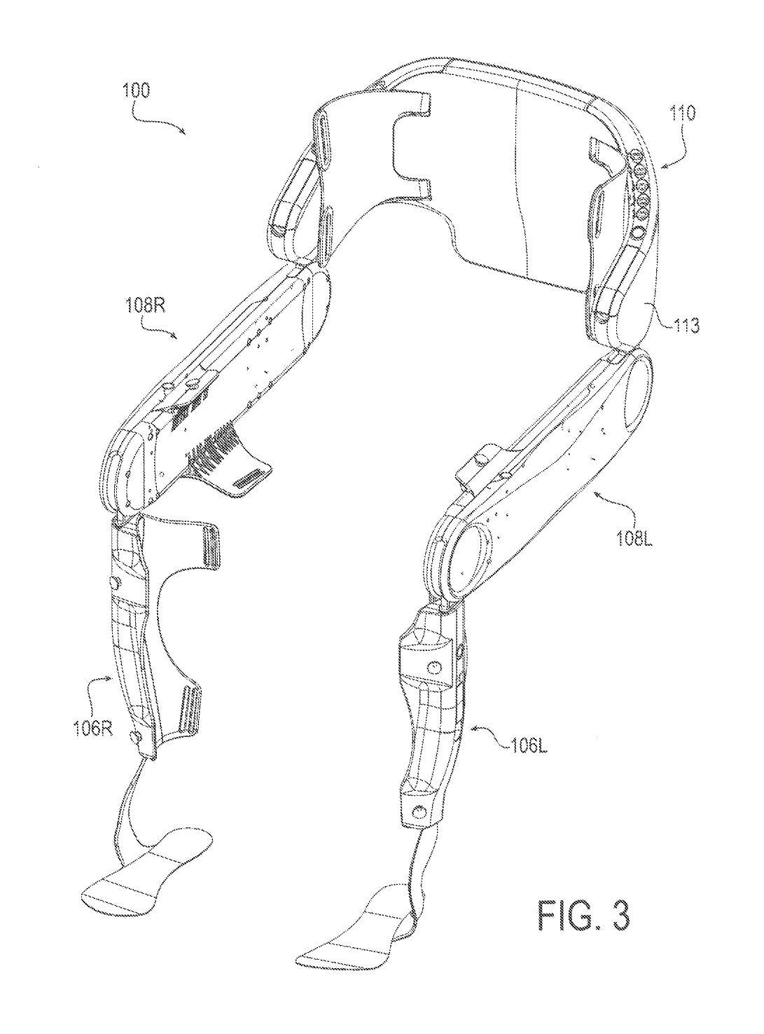

An exemplary legged mobility exoskeleton device is illustrated as a powered lower limb orthosis 100 in FIGS. 2-6. Specifically, the orthosis 100 shown in FIGS. 2-6 may incorporate four drive components configured as electro-motive devices (for example, electric motors), which impose sagittal plane torques at each knee and hip joint components including (right and left) hip joint components 102R, 102L and knee joint components 104R, 104L. FIG. 2 shows the orthosis 100 in a standing position while FIG. 3 shows the orthosis 100 in a seated position.

As seen in the figures, the orthosis contains five assemblies or modules, although one or more of these modules may be omitted and further modules may be added (for example, arm modules), which are: two lower (right and left) leg assemblies (modules) 106R and 106L, two (left and right) thigh assemblies 108R and 108L, and one hip assembly 110. Each thigh assembly 108R and 108L includes a respective thigh assembly housing 109R and 109L, and link, connector, or coupler 112R and 112L extending from each of the knee joints 104R and 104L and configured for moving in accordance with the operation of the knee joints 104R and 104L to provide sagittal plane torque at the knee joints 104R and 104L.

The connectors 112R and 112L further may be configured for releasably mechanically coupling each of thigh assembly 108R and 108L to respective ones of the lower leg assemblies 106R and 106L. Furthermore, each thigh assembly 108R and 108L also includes a link, connector, or coupler 114R and 114L, respectively, extending from each of the hip joint components 102R and 102L and moving in accordance with the operation of the hip joint components 102R and 102L to provide sagittal plane torque at the knee joint components 104R and 104L. The connectors 114R and 114L further may be configured for releasably mechanically coupling each of thigh assemblies 108R and 108L to the hip assembly 110.

In some embodiments, the various components of device 100 can be dimensioned for the user. However, in other embodiments the components can be configured to accommodate a variety of users. For example, in some embodiments one or more extension elements can be disposed between the lower leg assemblies 106R and 106L and the thigh assemblies 108R and 108L to accommodate users with longer limbs. In other configurations, the lengths of the two lower leg assemblies 106R and 106L, two thigh assemblies 108R and 108L, and one hip assembly 110 can be adjustable. That is, thigh assembly housings 109R, 109L, the lower leg assembly housings 107R and 107L for the lower leg assemblies 106R, 106L, respectively, and the hip assembly housing 113 for the hip assembly 110 can be configured to allow the user or medical professional to adjust the length of these components in the field. For example, these components can include slidable or movable sections that can be held in one or more positions using screws, clips, or any other types of fasteners. In view of the foregoing, the two lower leg assemblies 106R and 106L, two thigh assemblies 108R and 108L, and one hip assembly 110 can form a modular system allowing for one or more of the components of the orthosis 100 to be selectively replaced and for allowing an orthosis to be created for a user without requiring customized components. Such modularity can also greatly facilitate the procedure for donning and doffing the device.

In orthosis 100, each thigh assembly housing 109R, 109L may include substantially all the drive components for operating and driving corresponding ones of the knee joint components 104R, 104L and the hip joint components 102R, 102L. In particular, each of thigh assembly housings 109R, 109L may include drive components configured as two motive devices (e.g., electric motors) which are used to drive the hip and knee joint component articulations. However, the various embodiments are not limited in this regard, and some drive components can be located in the hip assembly 110 and/or the lower leg assemblies 106R, 106L.

A battery 111 for providing power to the orthosis can be located within hip assembly housing 113 and connectors 114R and 114L can also provide means for connecting the battery 111 to any drive components within either of thigh assemblies 108R and 108L. For example, the connectors 114R and 114L can include wires, contacts, or any other types of electrical elements for electrically connecting battery 111 to electrically powered components in thigh assemblies 108R and 108L. In the various embodiments, the placement of battery 111 is not limited to being within hip assembly housing 113. Rather, the battery can be one or more batteries located within any of the assemblies of orthosis 100.

The referenced drive components may incorporate suitable sensors and related internal electronic controller or control devices for use in control of the exoskeleton device. Such internal control devices may perform using the sensory information the detection of postural cues, by which the internal control device will automatically cause the exoskeleton device to enter generalized modes of operation, such as sitting, standing, walking, variable assist operation, and transitions between these generalized modes or states (e.g., Sit to Stand, Stand to Walk, Walk to Stand, Stand to Sit, etc.) and step transition (e.g., Right Step, Left Step). The internal electronic control devices further may perform fall mitigation and recovery operations for the exoskeleton device, as described for example in Applicant's International Patent Appl. No. PCT/US2016/016319 filed on Feb. 3, 2016, which is incorporated here in its entirety by reference.

The internal electronic control devices and related electronics further may incorporate or include a mobility device communications interface that is configured to transmit and receive signals over an electronic signal interface. In exemplary embodiments as further detailed below, the mobility device communications interface may communicate electronically over a wireless interface by transmitting signals to and receiving signals from a communications interface of an electronic communication device including a control application for controlling the drive components of the mobility device.

To perform such operations, the drive systems and internal control device of the mobility device may employ the use of accelerometers, gyroscopes, inertial measurement, and other sensors to detect and observe the upper leg orientation or angle and angular velocity. The internal control device may then selectively control the drive components to modulate the joint components, and particularly the knee and hip joint components, to apply torque, implement locked or released states, or otherwise effect positioning or movement of the joint components control of the exoskeleton device for mode operation or for fall mitigation.

To implement the features of the present invention, the electronic control device may include one or processor devices that are configured to execute program code stored on a non-transitory computer readable medium embodying the control methods associated the generalized control of the exoskeleton device, including the control operations of the present invention. It will be apparent to a person having ordinary skill in the art of computer programming of electronic devices how to program the electronic control device to operate and carry out logical functions associated with present invention. Accordingly, details as to specific programming code have been left out for the sake of brevity. Also, controller functionality could be carried out via dedicated hardware, firmware, software, or any combinations thereof, without departing from the scope of the invention. As will be understood by one of ordinary skill in the art, therefore, the electronic control device may have various implementations. For example, the electronic control device may be configured as any suitable processor device, such as a programmable circuit, integrated circuit, memory and I/O circuits, an application specific integrated circuit, microcontroller, complex programmable logic device, other programmable circuits, or the like. The electronic control device may also include a non-transitory computer readable medium, such as random access memory (RAM), a read-only memory (ROM), an erasable programmable read-only memory (EPROM or Flash memory), or any other suitable medium. Instructions for performing the methods described below may be stored in the non-transitory computer readable medium and executed by the processor device.

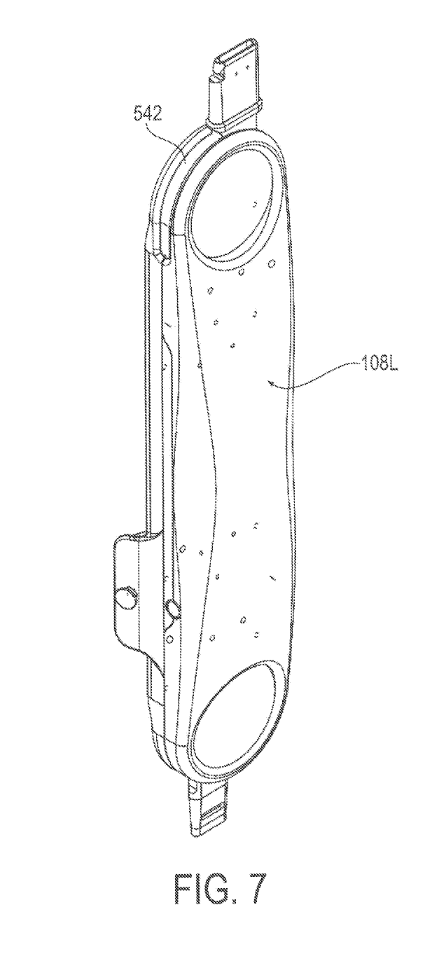

In the various embodiments, to maintain a low weight for orthosis and a reduced profile for the various components, the drive components may include a substantially planar drive system that is used to drive the hip and knee articulations of the joint components. For example, each motor can respectively drive an associated joint component through a speed-reduction transmission using an arrangement of sprocket gears and chains substantially parallel to the plane of sagittal motion. Referring to FIGS. 7-13, consolidating the moveable parts into self-contained units, referred to herein as “cassettes,” allow for ease of maintenance and replacement because cassettes are swappable, making them easier to service or requiring less of a variety in spare components. As used herein, “self-contained” means that the cassette includes everything necessary to operate in a fully functional manner if supplied with power. Thus, for example, if power is supplied to electrical contacts of the cassette, the cassette would actuate.

In the illustrated embodiment of the drive components, the motor is integrated onto a common baseplate along with sprockets that control the motion of a joint link. Bearings and chains, with and/or without tensioners provide smooth and efficient transfer of motion from the motor to the joint angle. Integrating the motor into the cassette allows for a thinner overall package configuration and provides consistent alignment among parts. Moreover, integrating the motor also creates a larger surface area to transfer and emit heat generated by the motor. In the instance of a mobility assistance device, these cassettes may pertain to a specific joint or set of joints on the device. Each may have a unique actuation unit or share an actuation unit. They may include actuators, with or without a power source, and/or a method of transmitting movement. The illustrated embodiment includes a brushless DC motor with chains and sprockets to create and transmit motion, although other embodiments may utilize electric motors, linear actuators, piezoelectric actuators, belts, ball screws, harmonic drive, gear drive (bevel or planetary), or any combination thereof. The cassettes may also house the electronic control device, and further may contain the referenced sensor elements such as the accelerometers, gyroscopes, inertial measurement, and other sensors to detect and observe the upper leg orientation or angle and angular velocity. The self-contained cassette units can be preassembled to aid in manufacturing the broader device. This allows for quick servicing of the device since individual cassettes can be swapped out and serviced.

Therefore, a removable, self-contained, ovular actuator cassette 500 may be receivable in a receptacle of a wearable robotic device. The cassette 500 may include a first circular portion 520 housing a motive device (e.g., an electric motor) 502. A second circular portion 522 may be longitudinally offset and longitudinally overlapping the first circular portion and may house a first portion of a drivetrain 514, 516 operatively coupled to and driven by the motive device 502. A third circular portion 524 may be longitudinally offset from the first and second circular portions and longitudinally overlapping the second circular portion and may house a second portion of the drivetrain 504. These three overlapping circular portions make an ovular shape, which may include the referenced sensors and electronic control devices. Therefore, an ovular housing 530 may support the motive device 502 and drivetrain 502, 514, 516. Long sides of the ovular housing are straight and parallel with each other and tangentially terminate as curved end surfaces of the ovular housing.

Referring to FIGS. 7-13, with FIG. 13 of the right side being representative, the powered joints may be implemented by disposing a joint sprocket gear 504 at one end of thigh assembly housing 109R parallel to the sagittal plane and configuring the joint sprocket gear 504 to rotate parallel to the sagittal plane. To provide the sagittal plane torque for knee joint component 102R, the connector 112R can extend from the joint sprocket gear 504 and be mechanically connected, so that rotation of the joint sprocket gear 504 results in application of torque to the lower leg assembly 106. A slot or receiving element can be provided for the connector 112R to link the thigh assembly 108R and lower leg assembly 106R. The receiving element and the connector 112R can be configured such that the connector can removably connect the thigh assembly 108R and lower leg assembly 106R. In the various embodiments, clips, screws, or any other types of fastener arrangements can be used to provide a permanent or a removable connection. In some embodiments, quick connect or “snap-in” devices can be provided for providing the connection. That is, these quick connect devices allow connections to be made without the need of tools. These types of quick connect devices can not only be used for mechanically coupling, but for electrical coupling with the sensors and control electronics. In some embodiments, a single quick connect device can be used to provide both electrical and mechanical coupling. However, the various embodiments are not limited in this regard and separate quick connect devices can be provided for the electrical and mechanical coupling. It is worth noting that with quick disconnect devices at each joint, the orthosis can be easily separated into three or five modular components—right thigh, left thigh, right lower leg, left lower leg, and hip assemblies—for ease of donning and doffing and also for increased portability.

The knee joint component 104R may be actuated via operation of a motor 502, as discussed above. The motor 502 can be an electric motor that drives the knee joint 104R (i.e., joint sprocket gear 504) using a two-stage chain drive transmission. For example, as shown in FIG. 13, a first stage can include the motor 502 driving, either directly or via a first chain, a first drive sprocket gear 514. The first drive sprocket gear 514 is mechanically coupled to a second drive sprocket gear 516 so that they rotate together about the same axis based on the power applied by motor 502 to first drive sprocket gear 514. The second drive sprocket gear 516 can be arranged so that it is disposed in the same plane as the joint gear 504. Thus, a second chain can then be used to drive joint sprocket gear 504 using the second drive sprocket gear 516 and actuate the knee joint 104R. The gear ratios for the various components described above can be selected based on a needed amount of torque for a joint, power constraints, and space constraints.

Each stage of the chain drive transmission can include tensioners, which can remove slack from a chain and mitigate shock loading. Such tensioners can be adjustable or spring loaded. In addition, a brake 570 can be provided for motor 502. For example, a solenoid brake may be provided which engages a brake pad against the rotor 524 of the motor 502 in one state, and disengages the brake pad in another state. However, the various embodiments are not limited to this particular brake arrangement and any other methods for providing a brake for motor 502 can be used without limitation.

The configuration illustrated in FIG. 13 has been discussed above with respect to an arrangement of sprocket gears and chains. However, the various embodiments are not limited in this regard. That is, any other arrangement of gears, with or without chains, and providing a reduced profile can be used. Furthermore, the various embodiments disclosed herein are not limited to an arrangement of gears and/or chains. For example, in some configurations, a belt and pulley arrangement could be used in place of the chain and sprocket arrangement. Furthermore, a friction drive arrangement can also be used. Also, any combination of the arrangements discussed above can be used as well. Additionally, different joints can employ different arrangements.

In the various embodiments of the drive components, a motor for each of the hip and knee joint components 102R, 102L, 104R, 104L can be configured to provide a baseline amount of continuous torque and a higher amount of torque for shorter periods of time. For example, in one configuration, at least 10 Nm of continuous torque and at least 25 Nm of torque for shorter (i.e., 2-sec) durations are provided. In another example, up to 12 Nm of continuous torque and 40 Nm of torque for shorter (i.e., 2-sec) durations. As a safety measure, both knee joints 104R and 104L can include normally locked brakes, as discussed above, in order to preclude knee buckling in the event of a power failure.

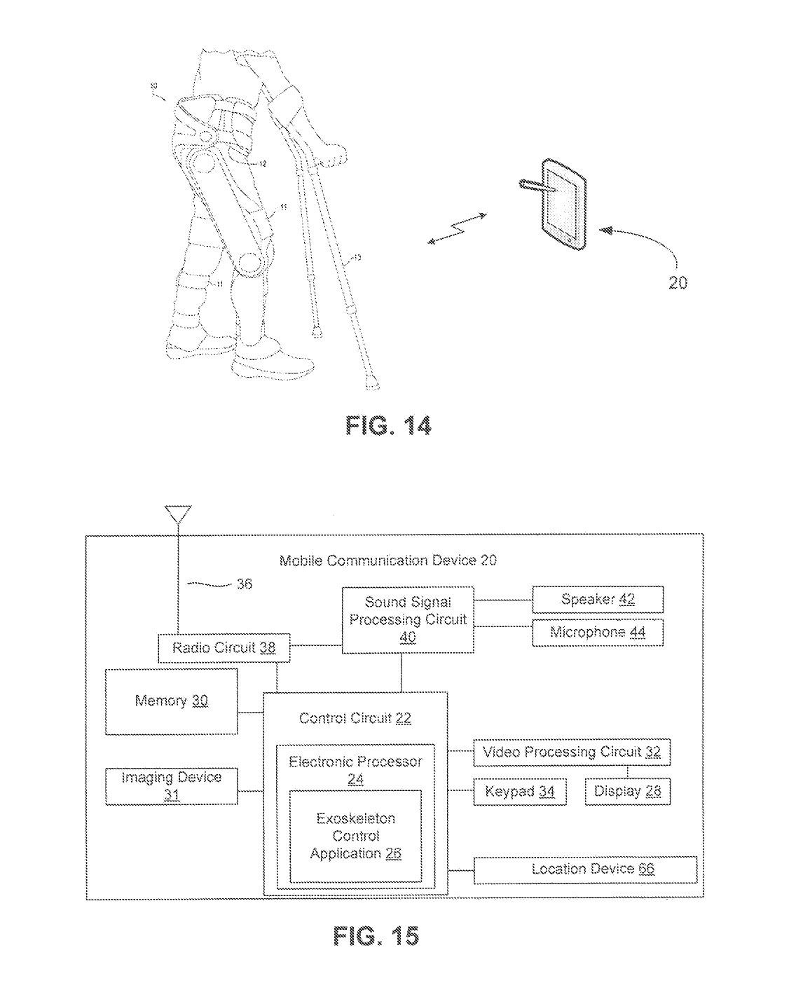

The control system of the present invention provides for additional external control of the exoskeleton device, the external control providing settings and commands that then may be implemented by the internal control devices and mechanisms described above. The control system of the present invention therefore may include one or more mobile communication devices 20. FIG. 14 is a drawing of a generalized depiction of an exemplary mobile communication device 20 interacting with the exoskeleton device 10 of FIG. 1. In the example depiction in FIG. 14, mobile communication device 20 is shown as being a tablet style computing device, but the invention is not limited to any particular electronic device. Rather, the mobile communication device 20 may be any portable electronic device with computing functionality as are known in the art. Examples of such devices include mobile telephones, smartphones, tablet or laptop computers, and like devices. Furthermore, although less convenient, the present invention may be implemented using a non-portable computer device, such as a desktop computer, where portability may not be an issue (such as in a permanent clinical setting or hospital). Using a mobile or portable communication device such as the device 20 of the example of FIG. 14 generally would be preferred.

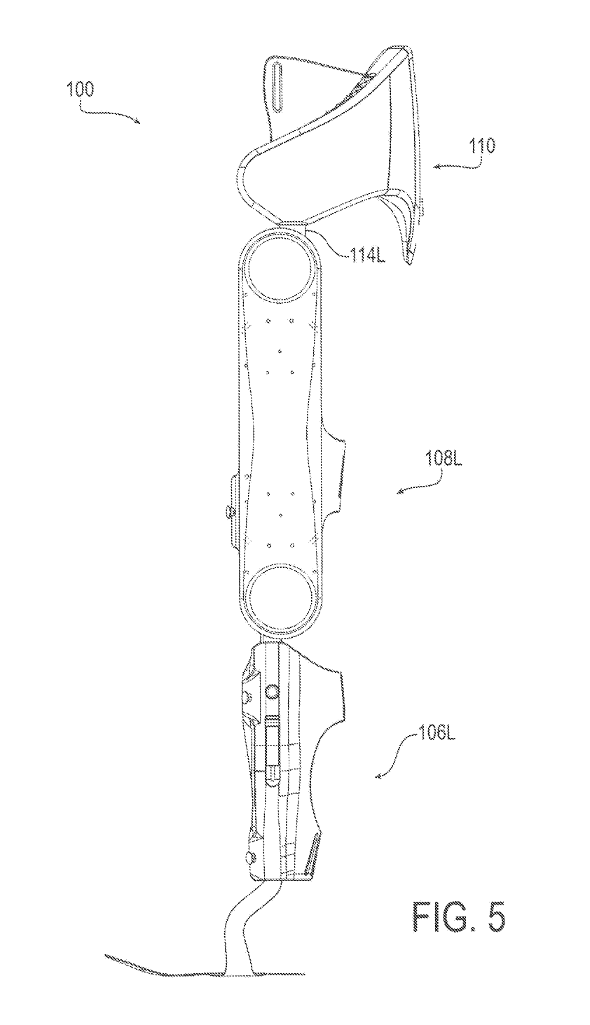

FIG. 15 is a schematic block diagram depicting operative portions of an exemplary mobile communication device 20 in accordance with embodiments of the present invention. The device 20 may include a primary control circuit 22 that is configured to carry out overall control of the functions and operations of the device. The control circuit 22 may include an electronic processor 24, such as a CPU, microcontroller or microprocessor. Among their functions, to implement the features of the present invention, the control circuit 22 and/or electronic processor 24 may comprise an electronic controller that may execute program code embodied as the exoskeleton control application 26. It will be apparent to a person having ordinary skill in the art of computer programming, and specifically in application programming for mobile electronic and communication devices, how to program the device to operate and carry out logical functions associated with application 26. Accordingly, details as to specific programming code have been left out for the sake of brevity. The control application 26 may be stored in a non-transitory computer readable medium, such as random access memory (RAM), a read-only memory (ROM), an erasable programmable read-only memory (EPROM or Flash memory), or any other suitable medium. In the example of FIG. 5, the control application 26 is shown as being stored internally within the processing components, but the application also may be stored in an additional memory device such as the memory 30. Instructions for performing the methods described below that are stored in the non-transitory computer readable medium may be executed by the processor components 22 and 24. Also, while the code may be executed by control circuit 22 or processor 24 in accordance with an exemplary embodiment, such controller functionality could also be carried out via dedicated hardware, firmware, software, or combinations thereof, without departing from the scope of the invention.

The mobile communication device has a display 28 that displays information to a user regarding the various features and operating state of device, and displays visual content received by the device and/or retrieved from a memory 30. Also, the display 28 may be used as an electronic viewfinder for a an imaging device 31, such as a camera assembly. Visual information is processed by a video processing circuit 32. The device further may have a keypad 34 that provides for a variety of user input operations. For example, keypad 34 typically includes alphanumeric keys for allowing entry of alphanumeric information. Keys or key-like functionality also may be embodied as a touch screen associated with the display 28. In the present invention, key-like functionality on the display may be particularly suitable for operating and executing the features of the exoskeleton control application 26. Due to the typical conditions of users of exoskeleton devices, the user interface exoskeleton control application 26 may be optimized for use by specific user populations, such as individuals having spinal cord injury, or who have experienced a cerebral vascular accident, by enabling broad touch-based or motion-based controls.

The device may include an antenna 36 coupled to a radio circuit 38. The radio circuit 38 includes a radio frequency transmitter and receiver for transmitting and receiving signals via the antenna 36 as is conventional in mobile communication devices. In this manner, the mobile communication device 20 may be connectable with other wireless communications devices over any suitable wireless network, such as for example, WiFi, cellular, Bluetooth, near field communication (NFC), passive and active RFID communication, and others. For near type communications that may employ scanning type interfaces (e.g., barcodes, QR codes), the imaging device 31 also may be used as a scanner for reading such coded information. The device further includes a sound signal processing circuit 40 for processing audio signals transmitted by and received from the radio circuit 38. Coupled to the sound processing circuit 40 are a speaker 42 and microphone 44 as is conventional for many mobile communication devices.

In exemplary embodiments, the mobile communication device 20 further may include a location device 46. The location device may be a GPS receiver and processor or comparable location device, that can calculate location data pertaining to the mobile communication device, including such features as spatial or geographical coordinates or similar location information, altitude, velocity, and the like as is commonly utilized in connection with location services.

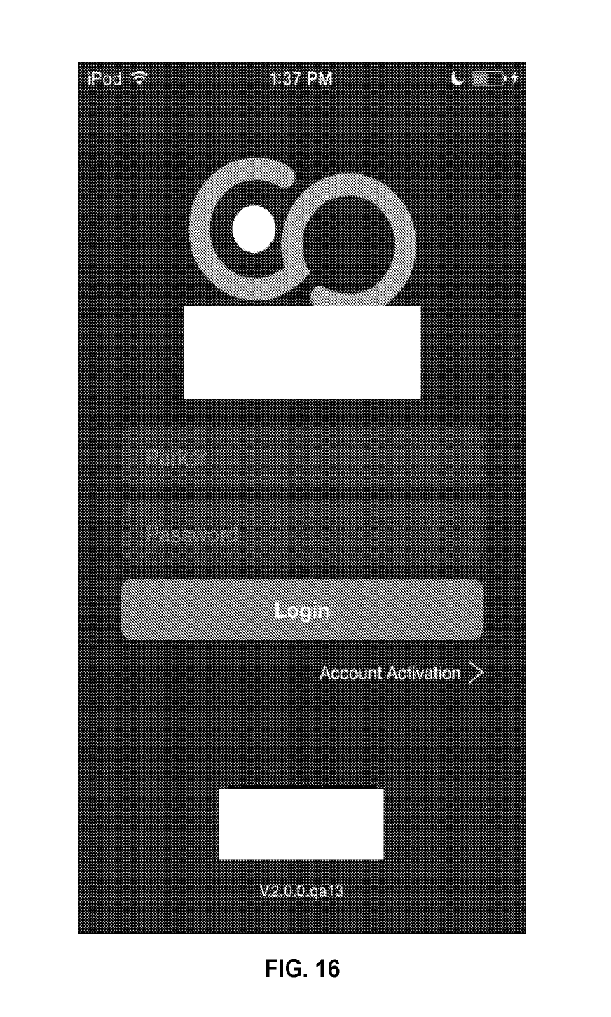

The following description relates to figures that are exemplary screenshots associated with execution of the exoskeleton control application 26 by the mobile communication device 20. It will be appreciated that the screenshots are examples intended to illustrate operation and features of the exoskeleton control application 26. The precise content and format of the displayed screens may be varied widely without departing from the scope of the present invention. In addition, the information and performance operations of the exemplary screenshots may be combined, separated, or otherwise organized in any suitable manner, and operations may be eliminated or added as may be appropriate for individualized circumstances, without departing from the scope of the present invention For initial implementation of the exoskeleton control application 26, FIGS. 16-18 are drawings depicting exemplary screenshots for generalized user access, setup, and identification.

In particular, FIG. 16 is a drawing depicting an exemplary screenshot for a user login screen in accordance with embodiments of the present invention. The exoskeleton control application 26 does not need to run on any dedicated or specialized hardware, but as referenced above may run on any suitable electronic device. Because of such versatility, the electronic application may be password protected to restrict access to application information and device function. In the example of FIG. 16, a user name and password may be entered, but any suitable mechanism for a secure login may be employed, including biometric data processing.

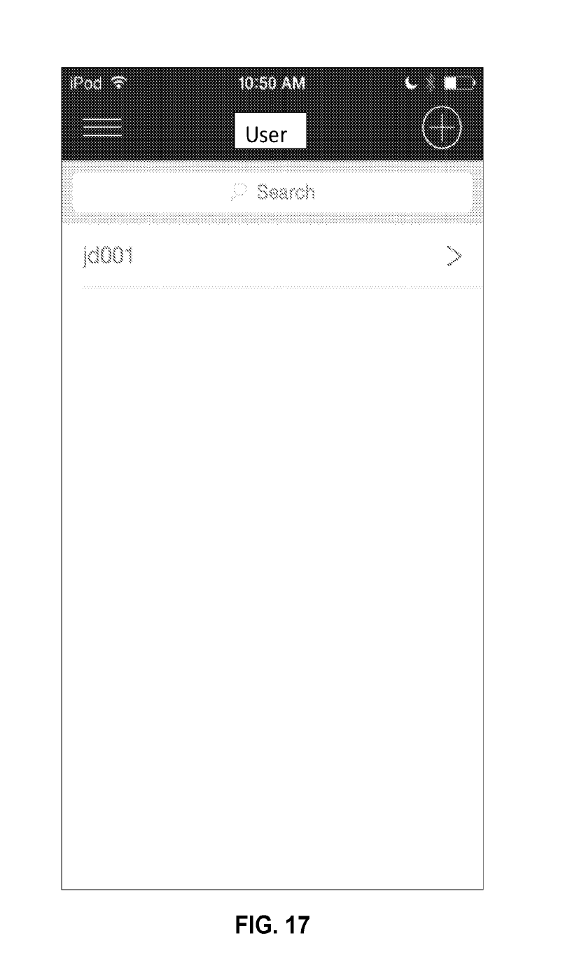

FIG. 17 is a drawing depicting an exemplary screenshot for a user accounts screen in accordance with embodiments of the present invention. The screenshot of FIG. 17 may arise after a login has been performed as described with respect to FIG. 16. The user accounts screen may provide a menu list of selectable individualized profiles for operation of an exoskeleton device or devices. At the top of the user accounts screen, a general name identifier (which may be the username from the login) may be shown for a user having access to the listed profiles. The name identifier is simply “User” in this case, but any suitable name identifier may be employed. One profile (jd001) is shown listed in the example profile list of FIG. 17, but any number of profiles may be listed and may be selected in any conventional manner for menu selections in electronic computer devices. The scope of the profile list may depend upon a scope of access associated with a given user login. For example, a clinician user may have access to profiles associated with multiple patients under the clinician's care and any associated exoskeleton devices. In contrast, a patient user would have more limited access to profile information only as to such patient's circumstances.

The user accounts screen also may include an option for creating a new profile. A profile may be created for a particular exoskeleton device, and/or for an individual person that may use an exoskeleton device. A profile also may be created for a use case, which may include parameters of a simulated situation. Simulated situations, for example, may include setting usages for different types of terrain. Other situational profiles may be based on a degree of assistance mobility, such as for example whether a user intends to utilize an assistance device (e.g., crutches or a walker) or not for someone who may have more partial mobility, as the device may operate differently for different levels of assistance. With the profiles, any given exoskeleton device may be rapidly configured for a given user or operational situation.

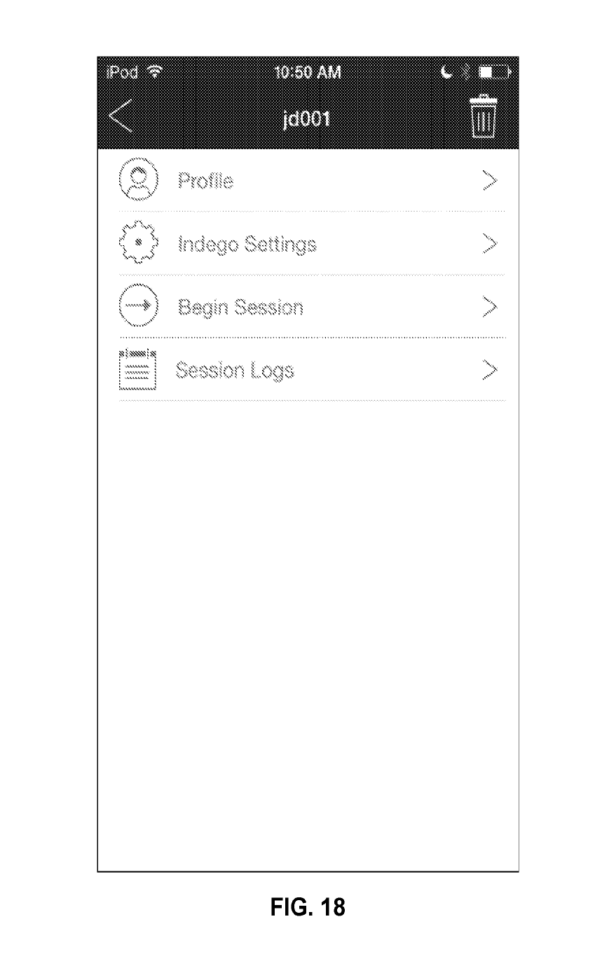

FIG. 18 is a drawing depicting an exemplary screenshot for a profile operation screen in accordance with embodiments of the present invention. The screenshot of FIG. 18 may arise upon selection of a particular profile that would have been listed in the user accounts screen of FIG. 17. For example, the screenshot of FIG. 18 provides access to profile jd001 that is in the list in FIG. 17. Generally, the profile operation screen may provide a menu list of options for exoskeleton device operation associated with such profile. In the example of FIG. 18, there are four exemplary menu options, which will each be explained in more detail below. Generally, a “Profile” menu option may permit accessing profile information for inputting and editing information pertaining to such profile. A “Device Settings” menu option may permit accessing device settings for inputting and editing settings for a particular exoskeleton device associated with the profile. A “Begin Session” menu option may permit initiating a session of use of the exoskeleton device. As referenced above, a session may encompass a specific time period of exoskeleton device use. A “Session Log” menu option may be used to export, store, and access recorded information pertaining to past sessions. It will be appreciated that these menu options represent examples, and additional and/or alternative menu options may be employed.

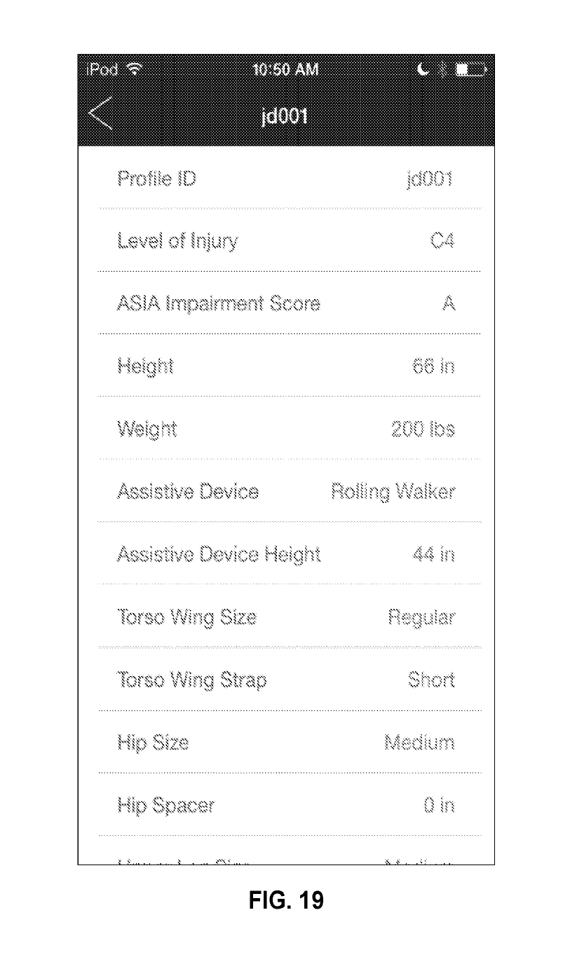

FIGS. 19-21 are exemplary screenshots pertaining to the menu options depicted in FIG. 18. In particular, FIG. 19 is a drawing depicting an exemplary screenshot for a Profile Detail screen in accordance with embodiments of the present invention. The Profile Detail screen may be shown upon selection of the Profile menu option from the screenshot of FIG. 18. The Profile Detail screen may provide any pertinent information relating to the selected profile. For example, the Profile Detail screen of FIG. 19 presents information about the user jd001, such as the user's level of injury, height, weight, etc., as seen in the figure. Other user based profile information may include a list of parameters relating to performance goals (improved session times, number of steps, or the like). The profile information may further include related exoskeleton device characteristics suitable for the user, such as torso wing size, hip size, etc., as also seen in the figure, as well as any related configuration parameters. It will be appreciated that any suitable information may be provided in the Profile Detail section. Additional user interface options may be provided for entering and editing any of the profile information items.

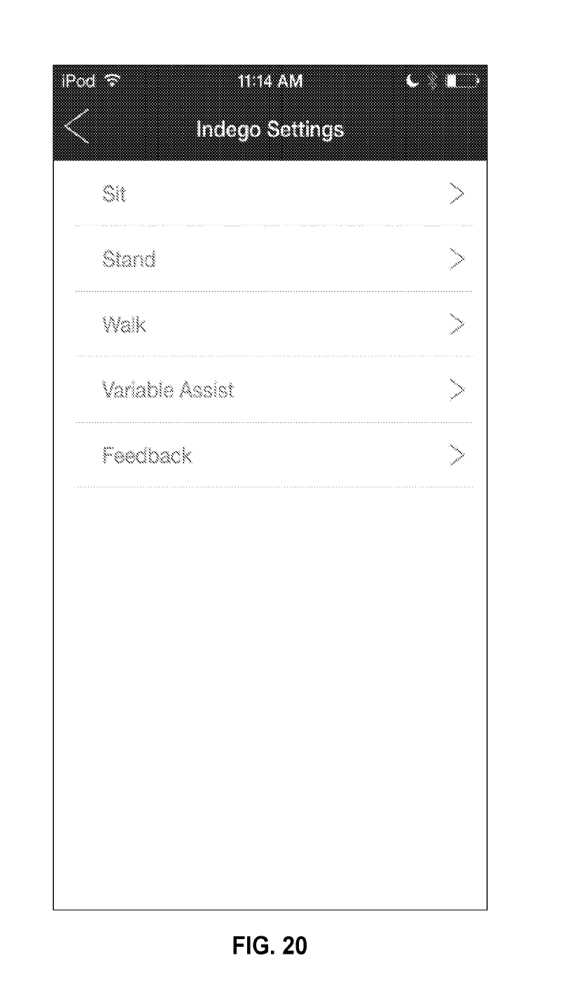

FIG. 20 is a drawing depicting an exemplary screenshot for a Device Settings screen in accordance with embodiments of the present invention. The Device Settings screen may be shown upon selection of the Settings menu option from the screenshot of FIG. 18. In one organizational example shown in FIG. 20, settings are grouped based on mode of operation, such as sitting, standing, and walking, which each constitutes a selectable menu option for a given category. Selection of any category will therefore permit a user to enter appropriate settings for a given mode of operation.

As referenced above, in the described exoskeleton device operation is automated based on sensory detections. As an example, to go from sit to stand a user may pull in the legs and lean forward, as any person normally does when getting ready to stand. Upon sensing such a pre-standing position, the exoskeleton drive system would send a feedback signal to the user, such as a vibration indicator, informing the user that a transition to standing will occur. The drive system will then operate the hip and knee joints for the user to stand. Settings, therefore, may include for example leg angle, torso tilt, a level of feedback, and the like for the user to perform the transition from sit to stand, and vice versa. Walking settings may include gait characteristics, such as step height, stride length, and the like. Settings also may include a “variable assist” category to enter an assistance level. Variable assist categories may relate to whether a subject uses and assistance device (e.g., walker, crutches, none), or relatedly may relate more generally to a subject's capability. For example, an exoskeleton user may be able to do one portion of tasks (e.g., 30%) without an assistance device, while doing a greater portion of tasks (e.g., 70%) with an assistance device. These degrees of capability may be reflected in the variable assist settings. There further may be default settings that a user may employ so as to simplify the settings process, i.e., a user may employ the default settings, or change only those settings most pertinent for the user.

Mode of operation (sit, stand, walk, etc.) further represents only one example of a settings organization. For example, an alternative method may be to have categories based on body component. For example, the menu list, instead of mode, could be leg angles, hip flexion, torso tilt, or the like, by which settings may be entered for such states for all modes (instead of entering the setting by mode). Other suitable settings organizations may be employed.

The device settings relatedly may include initial startup and configuration settings when a wireless connection is detected. For example, there may be a setting for enabling the exoskeleton device when a wireless connection is established with the mobile communication device that executes the exoskeleton control application. This may in turn result in an initial configuring of the exoskeleton device in accordance with the defined settings. While such wireless connection is established, the device settings may be changed and transmitted to the exoskeleton device. Any changed settings may be defined to take immediate effect, or delayed based on the state of the controlling mobile communication device and/or the state of the exoskeleton device.

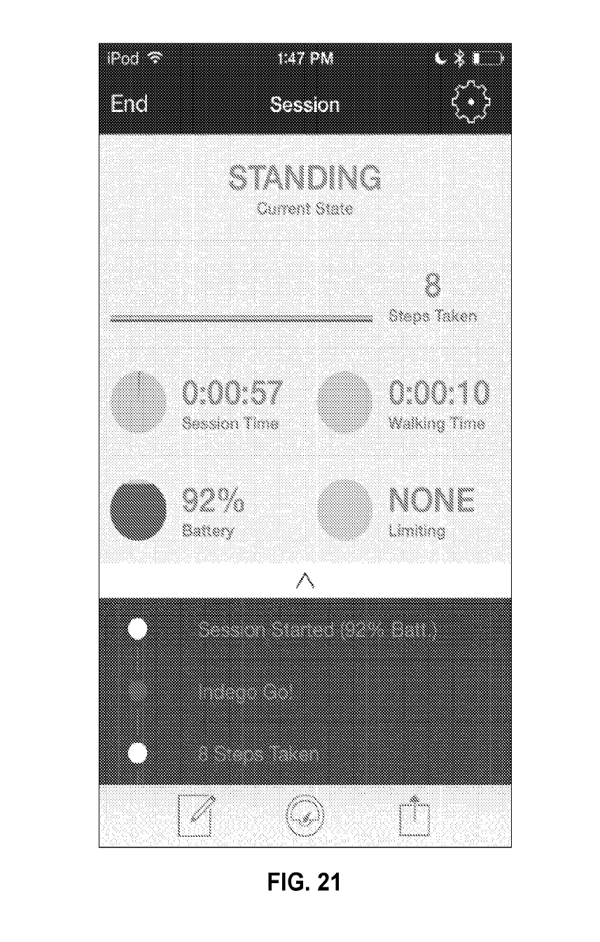

FIG. 21 is a drawing depicting an exemplary screenshot for a Session Dashboard screen in accordance with embodiments of the present invention. The Session Dashboard screen may be shown upon selection of the Begin Session menu option from the screenshot of FIG. 18. When a session is started, the mobile communication device by execution of the exoskeleton control application reads the stored settings and transmits the settings to the control components built into the exoskeleton device itself. The exoskeleton device control components will then configure the exoskeleton device as warranted, and as a user operates the exoskeleton device through the various modes of operation, the exoskeleton device control components will control the device operation in accordance with settings. Furthermore, information pertaining to the session will be gathered by the sensor and control components of the exoskeleton device, and such information may then be transmitted back to the mobile communication device executing the exoskeleton control application for display, storage, analysis, and other suitable processing.

In particular, such information may be displayed on the Session Dashboard screen on the portable electronic device, thereby permitting internal data relating to the exoskeleton device to be observed in the Session Dashboard. The Session Dashboard thus constitutes a real-time tracking screen for internal device data for an actual session of a user operating the exoskeleton device. Common information items on the Session Dashboard may relate to device operation events, such as for example session time, mode of operation (e.g., sitting, standing, walking), number of steps, walking speed, terrain, joint component states, and the like. Such aspects of the Session Dashboard particularly may be implemented or displayed as a scrolling screen viewed as a series of discrete events.

Other displayed information may be essentially an instrument panel for the exoskeleton device, including such information as battery level, device alerts, power consumption, software or firmware version, and the like. Instrument panel functionality through the Session Dashboard also may be employed to provide manual control of the exoskeleton device, including for example executing major mobility functions (e.g., stand, step) or specific joint function control (e.g., flex/extend a particular knee or hip joint). The scrolling events and the instrument panel type views may be displayed in combination or as separate viewing screens. Again, the Session Dashboard of FIG. 21 is an example, an any desirable information item pertaining to device use may be programmed to be display or selectable to be displayed in the Session Dashboard. Notable events also may be indicated, such for example meeting a goal or limit, a fall, a device component failure, or the like. Session information may be stored either automatically or by a specific user selection. In addition, session information may be stored and displayed locally on the mobile communication device executing the exoskeleton control application, or may be transmitted to another remote device for display and/or storage.

In a related manner, session information may provide real-time aid to the user. For example, GPS or comparable location data may be employed to configure the device based on a user location. As referenced above, the mobile communication device may include a GPS receiver and processor or comparable location device, that can calculate location data pertaining to the mobile communication device. In such embodiment, location data associated with corresponding device configurations may be inputted as part of the device settings described above. Location information also may be used to provide alerts or warnings of potential dangerous or “off-label” conditions in which device use is not recommended (e.g., wet or icy conditions). Such data further may be employed to interact with another external device. One example may be to open handicap doors when the user approaches. Such events may be indicated as items on the Session Dashboard as they occur.

The camera assembly of the mobile communication device also may be used to gather additional information, which further may be displayed as part of the

Session Dashboard. For example, a video recording of the exoskeleton device in operation may be synced to sensor data and overlaid in real-time onto the Session Dashboard in combination with related performance information. Barcode or other visual scanning may be performed with the mobile communication device camera to scan exoskeleton device components to call up additional device information for display on the Session Dashboard or perform other operations, such as for example display use history, request service, order components and determine compatibility, and the like all while the device is in use during a particular session.

The mobile communication device further may be used during a session as an extension of the sensor operation of the exoskeleton device. For example, the electronic device may provide for real-time sensor calibration or as a sensor itself. In an example of sensory extension, the mobile communication device can be placed on a component of the exoskeleton device, or on a stability aid, to enable new control features, such as leveraging a device accelerometer to change modes when a crutch is tapped on ground, or a light sensor can be used to determine and adjust LED brightness. Information as to such operations likewise may be indicated on the Session Dashboard.

Various categories of feedback also may be provided in the Session Dashboard. For example, the Session Dashboard may provide an on-screen graphical representation which mimics the physical exoskeleton device, which may be specific to a particular location of operation. Additionally, the graphical representation may mimic the motion or configuration of the physical exoskeleton device in real time. The Session Dashboard further may be transmitted for implementation and enhancement in a heads-up or virtual reality (VR) device display or in any suitable augmented reality environment. Enhanced audio features such as stereo feedback (e.g. Left/Right audio cues to indicate Left/Right step) may be used in combination with the Session Dashboard display to issue stereo audio cues to indicate information such as when to take a left or right step, or left or right step success, and the like.

The Session Dashboard further may be used in connection with enhanced communication options. When a mobile communication device (e.g., a smartphone type device) is used for executing the control application, voicemail, email, text alerts and other messages may be transmitted to notify the user of certain device conditions (e.g. if the exoskeleton device has not been used recently) or other parties (e.g., clinicians) of certain device conditions (e.g. if a sensor fault has just occurred). Communications functionality further permits enhanced integration with external and third party devices that may measure related health parameters during use of the exoskeleton device. Parameters measured by external devices may include for example heart rate, blood pressure, blood sugar, or other health parameters that can be used or recorded to provide another basis of controlling the exoskeleton device.

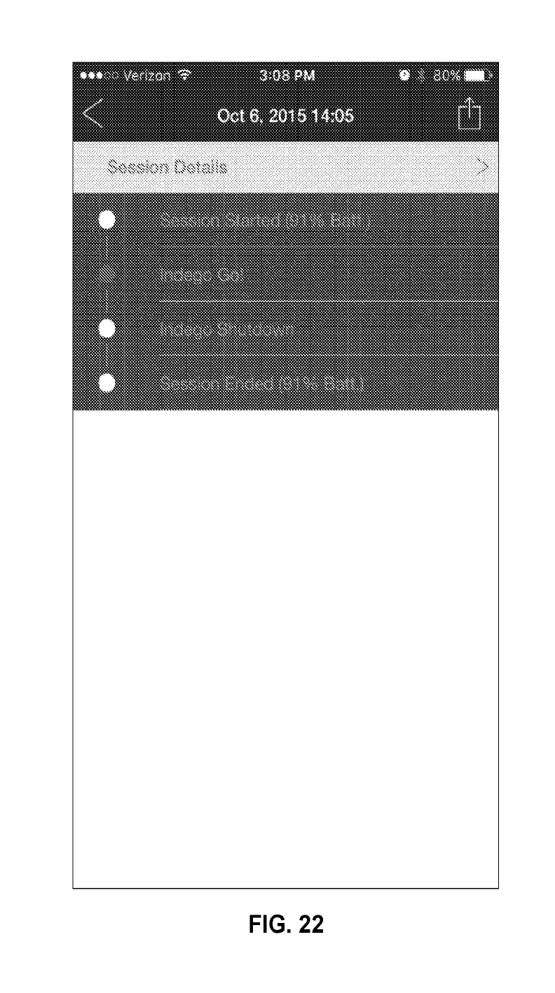

Referring back to FIG. 18, an additional selectable menu option may be a Session Logs option, which may be selected to access recorded information pertaining to past sessions. FIGS. 22-24 are exemplary screenshots pertaining to the different screens that may be displayed in connection with information accessible through the Sessions Logs option.

FIG. 22 is a drawing depicting an exemplary screenshot for a Session Log screen for an individual session in accordance with embodiments of the present invention. The example of FIG. 22 may display general information for a given session, such as session events and statistics (e.g., session time, number of steps, notable events). In exemplary embodiments, the session essentially may be replayed to simulate the real-time progression of the session.

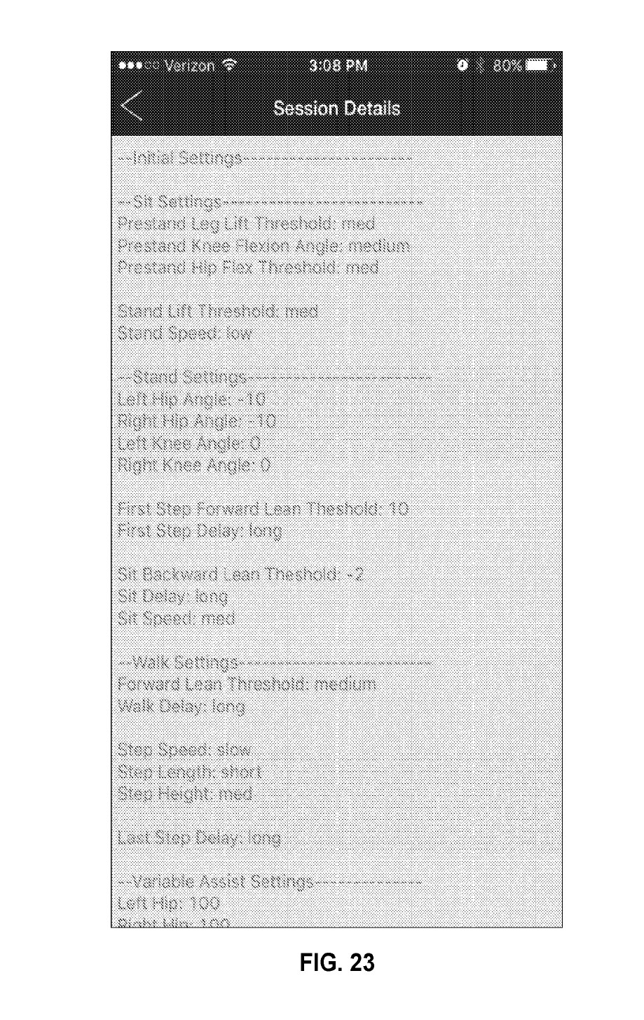

FIG. 23 is a drawing depicting an exemplary screenshot for a session details screen for an individual session in accordance with embodiments of the present invention. The screenshot of FIG. 23 may be displayed, for example, upon selection of a Session Details menu option or icon from the more general Session Log screen of FIG. 22. The Session Details screen may display more in-depth session information, such as for example corresponding device settings, software and firmware versions, device alerts or faults, performance details, and the like. Any desirable session information may be programmed to be incorporated into the Session Details screen.

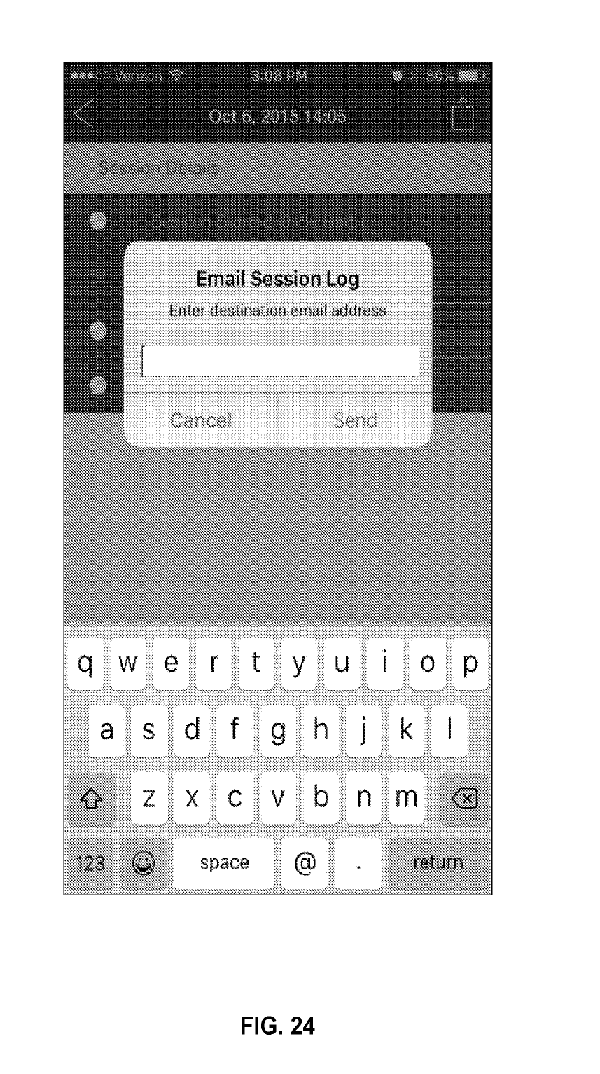

FIG. 24 is a drawing depicting an exemplary screenshot for a Session Log Export screen for an individual session in accordance with embodiments of the present invention. The screenshot of FIG. 24 may be displayed, for example, upon selection of an export menu option or icon from the more general Session Log screen of FIG. 22. The Session Log Export function may be used to transmit session information to an external storage device for access in the future. Session information may be exported manually or automatically via any suitable network, including various cloud services. Mobile communication devices that typically would execute the exoskeleton control application tend to have limited storage space. It, therefore, may be desirable to transmit session information for multiple sessions to a remote electronic device with more robust storage capabilities. In this manner, sessions that may be related, such as for example by user, exoskeleton device, or session date, may be collected in an associated fashion for a future complete analysis of all the related sessions. Gait analyses and historical performance trends may then be performed.

Accordingly, it is envisioned that the control application for the exoskeleton device may be employed in the context of a generalized therapeutic program for enhancing mobility. For example, the mobile communication device may automatically or manually report values through executing the exoskeleton control application to therapists' devices who may use such information to monitor a patient user remotely in either a one way to two communication. In a one-way communication, a therapist simply may be gathering performance information. In a two-way communication, a patient and therapist may interact directly through the exoskeleton control application for remote therapeutic benefit, diagnostics, and assistance.

As part of such a therapeutic program, performance goals (e.g., steps per time period, speed, terrain variation) may be set and monitored. The exoskeleton control application may be executed to track the progression toward those goals, and report when those goals have been completed. The exoskeleton control application may then, for example: (1) permit unlocking features of the exoskeleton device or the control application features or settings based on whether certain goals have been reached or proficiencies demonstrated; (2) may provide coaching type recommendations or advice as to how the user may best attain such goals; (3) provide motivational progress reports and motivational encouragement messages for display on the Session Dashboard (e.g. “You did it! 10,000 Steps this week!”); and (4) present and store past achievements as part of the session logs. It will be appreciated that these are examples, and any suitable therapeutic schemes or programs may be devised.

As another part of such a therapeutic program, various automated information services may be implemented. Examples may include automated collection of compliance data for device assessment, such as for example hours spent in the exoskeleton device or hours spent walking in the exoskeleton device. Another automated surface may be automated appointment scheduling based on device usage, such as for example based on whether specified milestones have been achieved (e.g. scheduling appointment every X Steps, or once X Speed/Hours achieved) or based on device conditions (e.g., scheduling appoint after sensor fault occurs.

Although the invention has been shown and described with respect to a certain embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A method of controlling a mobility device including at least one drive component that drives at least one joint component, the control method comprising the steps of:
providing said mobility device;
providing an electronic communication device having a control application to be executed by the electronic communication device;
receiving an input of settings information to the electronic communication device, the settings information being stored by the control application;
electronically connecting the electronic communication device to the mobility device; and
executing the control application with the electronic communication device to perform a session of using the mobility device, a session comprising information as to performance of the mobility device grouped according to a specific time period of device use;
wherein the electronic communication device executes the control application to control the at least one drive component of the mobility device to selectively configure and modulate the at least one joint component in accordance with the settings information.

2. The control method of claim 1, wherein the settings information comprises at least one selectable profile including settings associated with the profile;
the method further comprising receiving an input to the electronic control device selecting a profile, and the electronic communication device executes the control application to control the at least one drive component of the mobility device to selectively configure and modulate the at least one joint component in accordance with the settings associated with the selected profile.

3. The control method of claim 2, wherein the at least one selectable profile includes device information for a particular mobility device.

4. The control method of claim 2, wherein the at least one selectable profile includes user information for a user of the mobility device.

5. The control method of claim 2, wherein the at least one selectable profile includes a simulated situation of a use case for the mobility device.

6. The control method of claim 2, wherein the settings associated with the selected profile comprise settings organized by a plurality of modes of operation of the mobility device.

7. (canceled)

8. The control method of claim 2, wherein the settings associated with the selected profile comprise settings organized by joint component configuration of the at least one joint component.

9-10. (canceled)

11. The control method of claim 2, wherein the settings information includes a plurality of selectable profiles, and the method further comprises receiving an input to the electronic communication device selecting a profile from among the plurality of profiles.

12. The control method of claim 1, further comprising displaying session information pertaining to the session in real time on a session dashboard.

13. (canceled)

14. The control method of claim 12, wherein the session information comprises an instrument panel for the mobility device.

15. (canceled)

16. The control method of claim 14, wherein the session scrolling of discrete events and the instrument panel are displayed as a combined screen on the session dashboard.

17. The control method of claim 12, wherein the session dashboard is displayed on a display of the electronic communication device.

18-22. (canceled)

23. The control method of claim 12, further comprising storing the session information in a session log.

24. (canceled)

25. The control method of claim 23, further comprising storing a plurality of session logs for a plurality of sessions in a collection of associated sessions.

26. The control method of claim 1 wherein the electronic communication device includes a location device, and the control method further comprises acquiring location data during the session and controlling the mobility device based on the location data.

27. (canceled)

28. The control method of claim 1, wherein the electronic device connects to the mobility device over a wireless interface.

29. The control method of claim 1, wherein the mobility device is a legged mobility exoskeleton device comprising a plurality of drive components that drive a plurality of joint components including at least knee joint components and hip joint components.

30-31. (canceled)

32. A non-transitory computer readable medium storing program code for a control application for use in operating an electronic communication device to control a mobility device including at least one drive component that drives at least one joint component, the program code when executed by a computer performing the steps of:
receiving an input of settings information to the electronic communication device, the settings information being stored by the control application;
electronically connecting the electronic communication device to the mobility device; and
executing the control application with the electronic communication device to perform a session of using the mobility device, a session comprising information as to performance of the mobility device grouped according to a specific time period of device use;
wherein the electronic communication device executes the control application to control the at least one drive component of the mobility device to selectively configure and modulate the at least one joint component in accordance with the settings information.

33-59. (canceled)

60. An electronic communication device comprising:
an electronic controller that executes a control application for controlling a mobility device including at least one drive component that drives at least one joint component;
an input device for receiving an input of settings information to the electronic communication device;
a memory, the settings information being stored by the control application in the memory; and
a communications interface for electronically connecting the electronic communication device to the mobility device; and
wherein the electronic controller executes the control application to perform a session of using the mobility device, a session comprising information as to performance of the mobility device grouped according to a specific time period of device use;
wherein the electronic controller executes the control application to control the at least one drive component of the mobility device to selectively configure and modulate the at least one joint component in accordance with the settings information stored in the memory.

61-88. (canceled)

89. An exoskeleton system comprising:
the electronic communication device of claim 60; and
a mobility device comprising:
at least one drive component that drives at least one joint component;
a mobility device communications interface for electronically connecting the mobility device to the electronic communication device
an internal control device for controlling the at least one drive component to selectively configure and modulate the at least one joint component in accordance in accordance with signals received from the electronic device over the mobility device communications interface.

90-93. (canceled)
